(19) United States
(12) Patent Application Publication
(10) Pub. No.: US 2016/0324733 A1
(43) Pub. Date: Nov. 10, 2016

(54) Skin

(71) Applicant: Geana Jones, Pismo Beach, CA (US)

(72) Inventor: Geana Jones, Pismo Beach, CA (US)

(21) Appl. No.: 14/704,235

(22) Filed: May 5, 2015

(51) Int. Cl. A61K 8/02; A61Q 90/00; A61K 8/891

(52) U.S. Cl. CPC A61K 8/0208; A61K 8/891; A61Q 90/00

(57) ABSTRACT

An ultra-thin self-adhesive covering which effectively applied to an individual's skin that can block or hide existing tattoos or skin imperfections comprising: a semitransparent layer that prevents read through of the skin markings, at least one layer with one or more skin tone shade and or colors; at least one covering peel layer affixed to the back surface of said self-adhesive covering. In addition, said self-adhesive covering is semi-transparent, soft, flexible and highly reliable and can be layered for more coverage.

### CROSS REFERENCE TO RELATED APPLICATIONS

Not applicable.

### FEDERALLY SPONSORED RESEARCH AND DEVELOPMENT Not applicable.

### MICROFICHE APPENDIX

Not applicable.

### BACKGROUND OF THE INVENTION

(1) Field of the Invention

The present invention generally relates to layered structure for cosmetic use. More particularly, the present invention relates to an ultra-thin self-adhesive soft covering with tapered edges for an effective application to an individual's skin that can block or hide existing tattoos or skin imperfections.

(2) Background of Invention

A tattoo is a design on the skin that can be either permanent or non-permanent. Permanent tattoos are well known and have their origins dating back to at least as early as the Egyptian times. Permanent tattoos are typically applied to the skin with tattoo needles, which ink a desired design onto an individual's skin. Non-permanent tattoos are also known, and are typically adhered to an individual's skin in a variety of known fashions. Tattoos have been applied to an individual's skin for a variety of different purposes, including decoration, social status, and as a sign of endearment

While permanent tattoos have been around for thousands of years, the desire to remove or cover permanent tattoos has existed as long. The same desire that exists to remove or cover permanent tattoos also applies to scars or skin imperfections that may exist on an individual's skin. Typical processes for removing permanent tattoos or correcting or hiding scars and skin imperfections include painful and expensive surgical procedures that can result in scarring or skin tissue. Less permanent methods involve the application of make-up or other liquid concealers. The known make-up products that are used as temporary concealers suffer from a variety of disadvantages. For example, they are susceptible to smearing or smudging are not waterproof and therefore last only a relatively short period of time. Further, they require frequent reapplication to ensure that the individual's skin remains covered.

Additionally, some temporary tattoos are known to be utilized to cover up dark skin imperfections. However, these temporary tattoos have only limited effectiveness. For example, if these temporary tattoos are applied over a darker skin imperfection, the underlying skin imperfection will typically show through the temporary tattoo, which defeats the purpose of attempting to cover or hide it. In general, these current temporary tattoos include three discrete layers. The first layer is a temporary tattoo paint or design that is applied to paper. The second layer consists of a sticky substrate that is disposed on top of the tattoo paint or painted design to allow the above layer to adhere to the skin. The third layer consists of a clear plastic removable protective cover which is intended to protect the temporary tattoo patch.

An exemplary illustration of a prior method for covering a skin imperfection discloses a temporary tattoo for concealing veins, blemishes, wine marks, bruises, age marks and other skin conditions. The temporary tattoo disclosed therein consists of a transfer patch that is intended to match the skin tone and pigment holding an image of human skin. Thus, these temporary tattoos vary in tones in an effort to closely match and blend in with a variety of skin pigments and colorings. These temporary tattoos may also vary in size and shape to cover a particular skin imperfection. The temporary tattoo disclosed in consists of an opaque tattoo patch holding an image of human skin applied to translucent material, which is applied to conceal skin imperfections. While this temporary tattoo may work reasonably well on many skin imperfections it is ineffective in concealing a permanent tattoo there beneath. This is because an undertone of color from the permanent (or other temporary) tattoo will typically show through the temporary tattoo containing the image of the human skin. The same problem also applies with the application of this temporary tattoo to any high contrast or dark color skin imperfections. The end result is that despite the application of the opaque temporary tattoo, the permanent tattoo or skin imperfection is not completely concealed.

In addition various products were available basically comprise various types of cosmetics or makeup. Make-up is available in a wide range of colors designed to match different skin tones. However, the disadvantage of make-up is that it may be a problem for those who tend to sweat heavily, and for those who participate in strenuous physical activities. These types of activities can cause make-up to be easily smudged, wiped or rinsed off of the skin. Additionally, the existing products offer little coverage to an individual who wishes to go swimming, or to one who might get caught in the rain. In both situations, the make-up would be removed or smeared by the water. Also, make-up is easily rubbed off and may cause embarrassing stains to the clothing, while at the same time eliminating the benefits of the cover-up.

Other processes exist; however, they all also suffer from a variety of disadvantages. For example, some of the processes are expensive and therefore not readily available. Other processes do not adequately conceal dark skin imperfections or the ink from a permanent tattoo. Thus, a need exists for a temporary concealer for application to an individual's skin that will adequately hide all types of skin imperfections as well as permanent tattoos that is readily available, can be easily applied, and is relatively long lasting.

### BRIEF SUMMARY OF THE INVENTION

The present invention provides, an improved ultra-thin self-adhesive soft cover. The general purpose of the present invention is to provide a new and improved ultra-thin self-adhesive soft cover which has all the advantages of the prior art and none of the disadvantages.

The primary objective of the present invention is to provide an improved ultra-thin self-adhesive, matte finish cover in varying shades which can be effectively applied to an individual's skin that can block or hide existing tattoos or skin imperfections.

It is therefore, an advantage of the present invention to provide a self-adhesive tattoo cover for more efficiently concealing a permanent tattoo or other skin imperfection.

It is another advantage of the present invention to provide a temporary self-adhesive tattoo cover for concealing a permanent tattoo or other skin imperfection that prevents or blocks any “read through” of the underlying skin markings.

It is a further aim of the present invention to provide a sweat proof and waterproof skin concealing product that is not easily wiped or rinsed off.

It is another advantage of the present invention to provide a temporary self-adhesive cover which is semi-transparent in nature.

It is another advantage of the present invention to provide a temporary self-adhesive cover which is available in multiple skin tone shade or colors shades.

It is another advantage of the present invention to provide a temporary self-adhesive cover which is soft and flexible in nature.

It is another advantage of the present invention to provide a temporary self-adhesive cover having just peel and stick application.

It is a further aim of the present invention to provide a product that is realistically priced through the use of mass production.

It is still yet a further advantage of the present invention to provide a temporary self-adhesive cover for concealing a permanent tattoo or skin imperfection that is easy to apply and pain free.

It is another advantage of the present invention to provide a temporary self-adhesive cover for concealing a permanent tattoo or other skin imperfection that is washable and reusable.

In accordance with the above and other advantages of the present invention, a method for concealing permanent tattoos or other skin imperfections is provided. The method includes applying a self-adhesive cover to an individual's skin over a permanent tattoo or skin imperfection, which the individual wishes to conceal.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawings wherein;

FIG. 1 depicts an image of human skin as seen under the preferred self-adhesive covering of the present invention.

FIG. 2 depicts an image of ultra-thin self-adhesive soft cover for human skin.

FIG. 3 depicts an oval shape of the self-adhesive covering with dimensions.

### DETAILED DESCRIPTION OF THE INVENTION

In the following detailed description, reference is made to the accompanying figures which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description is, therefore, not to be taken in a limiting sense, and the scope of the present invention is defined by the appended claims and their equivalents.

In accordance with existing methods, skin imperfections are sometimes covered with a temporary tattoo having an image of skin with a color that matches the skin tone of the individual who seeks to cover their skin imperfection. However, when the temporary tattoo is positioned on top of the skin imperfection, the skin imperfection, while being fully covered, is not fully blocked and can still be observed through the temporary tattoo. This is, at least in part, because the temporary tattoo holding the image of skin does not sufficiently block the light reflecting from the dark skin imperfection. Thus, an observer can visually perceive the dark skin imperfection through the opaque temporary tattoo, a condition known as “read through.” The dark skin imperfection most likely appears as a shaded or muted undertone to the temporary tattoo. In some instances, it is possible that the details of the dark skin imperfection will “read through” the temporary tattoo such that both the dark skin imperfection and the temporary tattoo can be perceived. For example, the dark skin imperfection can undesirably read through the skin colored opaque temporary tattoo of the known prior art and appears as a muted dark or gray tone. These drawbacks are overcome by the invention discussed below and shown in the attached figures.

The basic patch is made from 5-20 durometer medical grade silicone rubber. The side next to the skin is coated with medical grade silicone gel which acts as a reusable adhesive (20-40 uses).

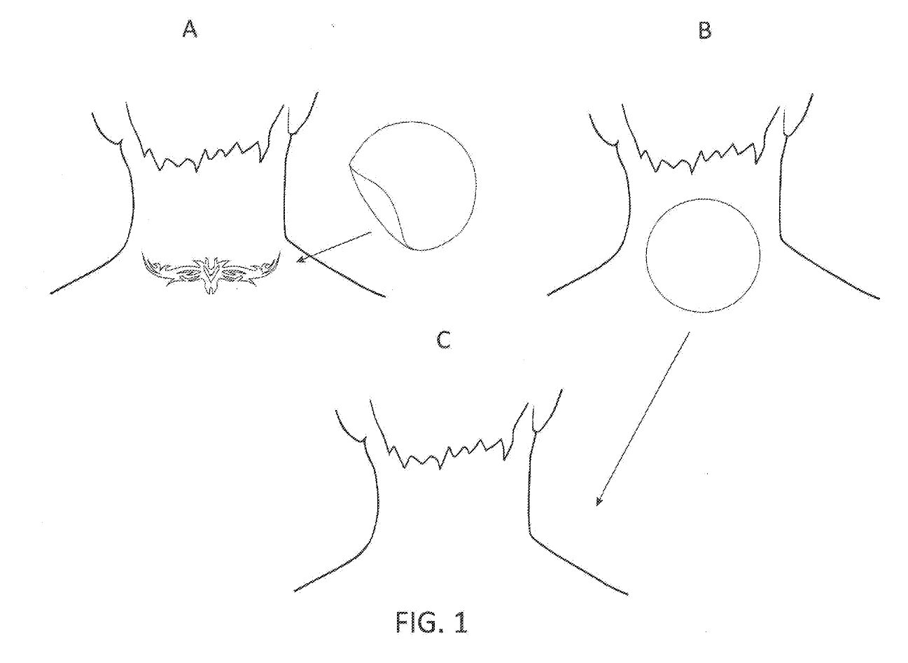

Referring now to FIG. 1 A illustrates the example of the tattoo and self-adhesive embodiments of a self-adhesive covering which is placed on the skin to cover the tattoo as it shown on FIG. 1 B. The disclosed ultra-thin self-adhesive covering is particularly usable on the skin of an individual for covering and concealing a permanent tattoo or other existing skin imperfection. Also, advised covering is matte finished with tapered edges for precut application. The temporary tattoo preferably consists of multiple layers or components. While the term layers are utilized, it will be understood that the layer can be combined or separated as desired and cover can be formed in a variety of different shades to cover the tattoo completely. Moreover, the ultra-thin self-adhesive covering may consist of a variety of different shapes and sizes. The self-adhesive covering may also be constructed in a multitude of color tones and pigments and. The variety of colors and shapes allows to create the effect when the self-adhesive covering is invisible as it shown on FIG. 1 C. Further, the preferred self-adhesive covering is waterproof or water resistant and sweats proof such that it does not wear off in water. Additionally, the disclosed temporary self-adhesive covering has increased longevity over other covering methods,

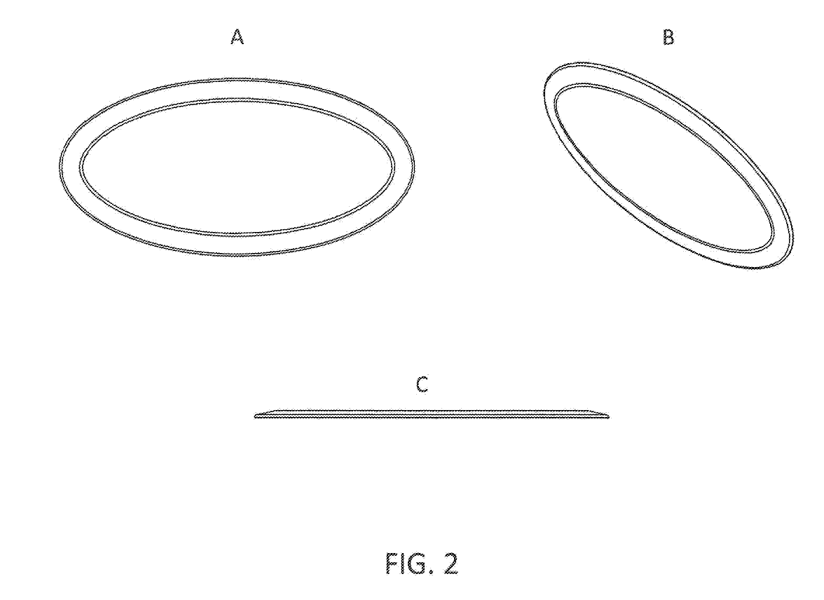

FIG. 2 illustrates an example of an embodiment of a temporary self-adhesive covering in accordance with the present invention where the layers cover the tattoo. The temporary tattoo may have a variety of shapes, sizes, color tones and pigments. As shown, where the layers consist of patches, the self-adhesive covering includes a clear protective cover with skin color shade. The clear protective covers protect the temporary tattoo from damage.

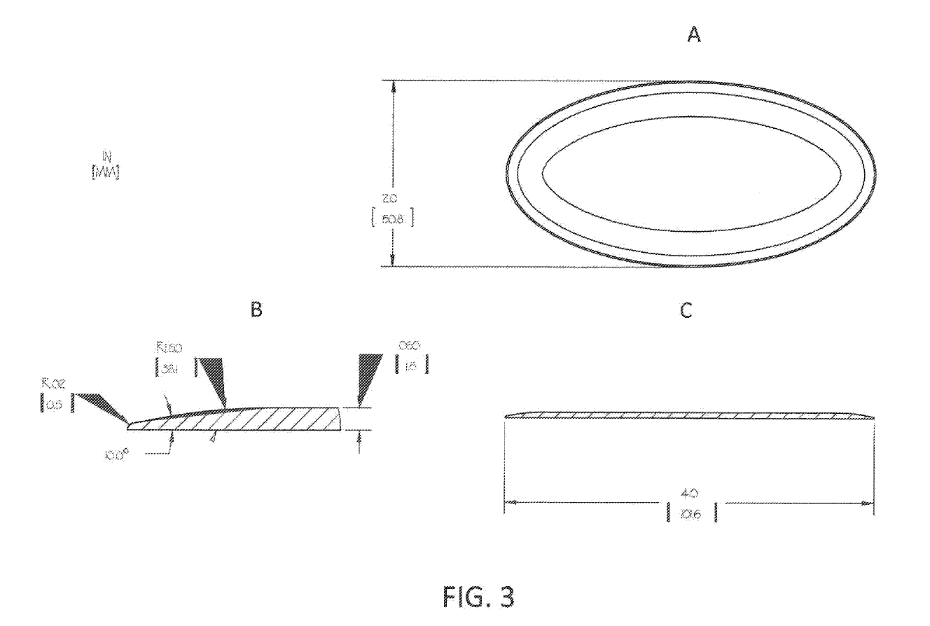

FIG. 3 illustrates the sized and configuration of the self-adhesive covering. As at shown in section B of the FIG. 3, the covering with tapered edges is an oval with dimensions 100.6 mm and 50.8 mm.

In general, the temporary self-adhesive covering includes a color layer and an adhesive layer with tapered edges which helps secure the temporary self-adhesive covering to the individual's skin. As is discussed in more detail, each of these layers can be applied individually or as multiple separate steps as shown in FIG. 2 this covering is washable preferably up to 30 times and reusable up to 40 times if applied properly. Additionally, the temporary self-adhesive covering can be made of silicon soft ultra-thin material which makes it soft and flexible for places that bend. In Addition, these self-adhesive covering can be performed or can be produced in a sheet and cut down to size as desired.

In another embodiment manufacturing self-adhesive covering includes tapered edges for precut application which is mate finished. It is preferably made of hypo allergic, medical grade, semi-transparent, silicone based material which is sterile for application. its use involves just peel and stick application which is used 40 times if applied properly.

Advantageously, self-adhesive covering as discussed above is semi-transparent in nature. Moreover, it is soft and flexible enough for placing it bend on the body. Also, it is simple in structure, easy to use, pain free, fairly inexpensive, and highly reliable and can be layered for more coverage.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. it is contemplated that these variations, modifications, additions and improvements fall within the scope of the invention.

1. A ultra-thin self-adhesive washable reusable covering which effectively applied to an individual's skin that can block or hide existing tattoos or skin imperfections comprising:
a semi-transparent layer preventing read through of the skin markings;
at least one layer with one or more skin tone shade and or colors; and
at least one covering peel layer affixed on back surface of said self-adhesive covering.

2. The ultra-thin self-adhesive washable reusable covering of claim 1, wherein said semi-transparent layer is matte finished.

3. The ultra-thin self-adhesive washable reusable covering of claim 1, wherein said skin tone shade and or color concealing a permanent tattoo or other skin imperfection through marking and blend more naturally with the individual's skin

4. The ultra-thin self-adhesive washable reusable covering of claim 1, wherein said cover is reusable up to 40 times.

5. The ultra-thin self-adhesive washable reusable covering of claim 4, wherein said cover is washable up to 30 times. The ultra-thin self-adhesive washable reusable covering of claim 1, wherein said cover layer is sweat proof.

7. The ultra-thin self-adhesive washable reusable covering of claim 1, wherein said cover is cut to fit for all sizes.

8. A ultra-thin self-adhesive washable reusable covering in its manufacturing comprising:
at least water proof, at least sweat proof, at least Hypo allergic, medical grade material for cosmetic purpose.

9. The ultra-thin self-adhesive washable reusable covering of claim 1, wherein said covering made of from 5-20 durometer medical grade silicone rubber material.
